(19) United States
(12) Plant Patent Application Publication
(10) Pub. No.: US 2004/0139512 P1
(43) Pub. Date: Jul. 15, 2004

(54) AGERATUM PLANT NAMED 'AGMONTIS'

(76) Inventor: Monica Sanders, Grootebroek (NL)

Correspondence Address: SYNGENTA BIOTECHNOLOGY, INC., PATENT DEPARTMENT, 3054 CORNWALLIS ROAD, P.O. BOX 12257, RESEARCH TRIANGLE PARK, NC, 27709-2257, US

(21) Appl. No.: 10/340,299

(22) Filed: Jan. 10, 2003

(51) Int. Cl. A01H 5/00

(52) U.S. Cl. PLT/263

(57) ABSTRACT

A new Ageratum plant particularly distinguished by its purple flower, early flowering, strongly branched plant habit and good growth.

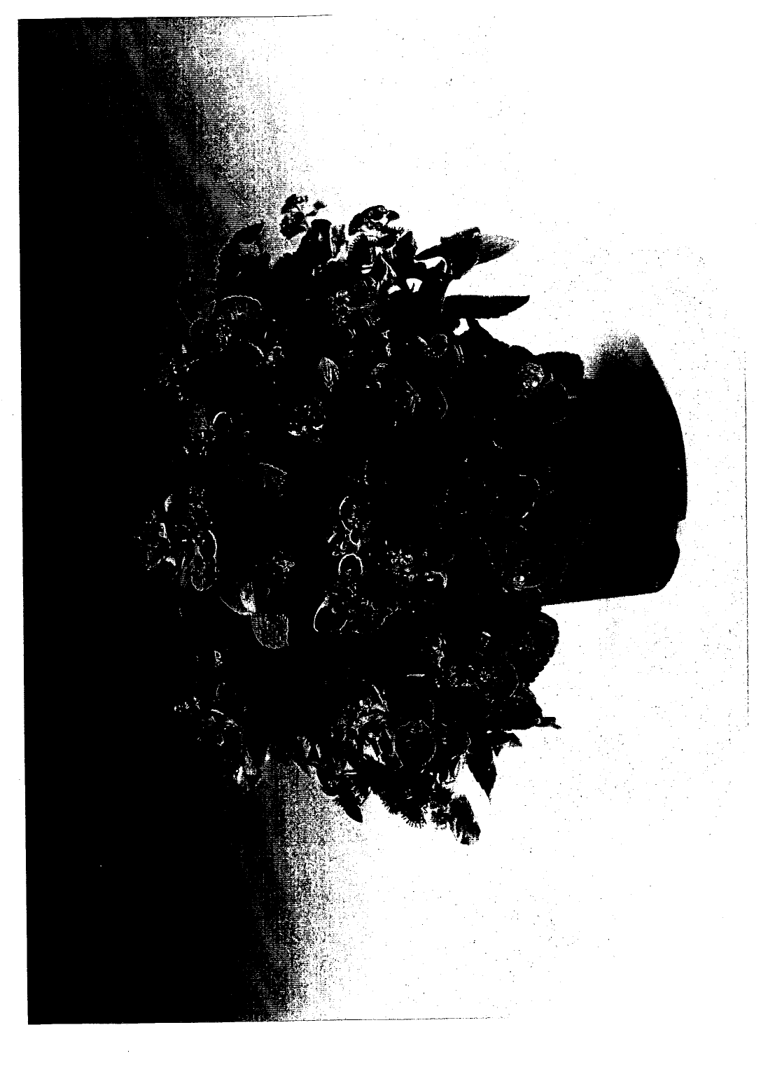

### VARIETAL DENOMINATION

‘Agmontis’

### BACKGROUND OF THE NEW PLANT

The present invention comprises a new distinct cultivar of Ageratum, botanically known as Ageratum houstonianum. The new cultivar is propagated by cuttings resulting from the cross of ‘S509-2’ and pollen from a population of S-labeled plants. ‘S509-2’ is a purple flowering Ageratum having a compact habit. ‘S509-2’ is not commercially available and is not known by any synonyms.

‘S509-2’ has not been patented. As a result of this cross the present cultivar was created in 1998 in Enkhuizen, Netherlands and has been repeatably asexually reproduced by cuttings in Enkhuizen, Netherlands, in Gilroy, Calif., United States, in Angers, France and in Sarrians, France over a three-year period. It has been found to retain its distinctive characteristics through successive propagations, and this novelty appears to be firmly fixed.

This new Ageratum plant is an annual in most climactical zones in the United States, only in zones 9 and 10 it is a perennial plant.

### DESCRIPTION OF THE DRAWING

This new Ageratum plant is illustrated by the accompanying photographic drawing which shows blooms, buds and foliage of the plant in full color, the color shown being as true as can be reasonably obtained by conventional photographic procedures.

### DESCRIPTION OF THE NEW CULTIVAR

The following detailed descriptions set forth the distinctive characteristics of this new Ageratum plant. The data which defines these characteristics were collected from asexual reproductions carried out in Enkhuizen, Netherlands. The plant history was taken on 30 weeks old plants, blossomed under natural light and artificial light in a greenhouse. Color readings were taken in a laboratory under ambient light. Color references are primarily to the RHS Colour Chart of The Royal Horticultural Society of London.

Comparison between the new cultivar ‘Agmontis,’
its parent and a similar cultivar

| | ‘Agmontis’ | ‘S509-2’ | ‘R116’ |
|---|---|---|---|
| Flower color | Purple | Violet | Blue |
| Size of capitulum | 4-8 mm | 4-8 mm | 7-9 mm |
| Plant height | 20-25 cm | 20-25 cm | 20-25 cm |
| Diameter of plant | 35-40 cm | 30-35 cm | 15-20 cm |

The plant:

Classification.—Botanical: Ageratum houstonianum.

Parentage:

Female parent.—A seedling named ‘S509-2’ is one of our seedings from our S-generation of plants bred in 1987.

Pollen parent.—A population of S-labeled plants.

Plant description:

Growth habit.—Ascending, well branched.

Plant height.—20-25 cm.

Speading area of plant.—35-40 cm.

Growth rate.—Compact.

Strength.—Very good.

Branching character.—Freely branching and lateral branching at every node.

Blooming period.—Year round.

The stem:

Diameter.—1-2 mm.

Shape.—Round.

Color.—RHS 145A.

Anthocyan pigmentation.—Absent.

Length of internode.—10-20 mm, depending on the light where the plant is propagated.

Pubescence.—Slightly pubescent.

The foliage:

Phyllotaxis.—Opposite, decussate.

Shape of blade.—Cordate.

Texture.—Upper side: Slightly pubescent — Lower side Slightly pubescent.

Venation.—Reticulate.

Leaf margin.—Serrate.

Leaf base.—Cordate.

Leaf apex.—Rounded.

Length.—18-25 mm.

Width.—15-25 mm.

Color.—Upper side: Dark green 137B — Lower side: Medium green 138B.

Pubescence.—Some pubescence is present.

Length of petiole.—3-6 mm.

Color of petiole.—Light green 145A.

Petiole surface texture.—Slightly pubescent.

Inflorescence:

Inflorescence.—Compound umbel of capitula.

Length of peduncle.—10-20 mm.

Color of peduncle.—RHS 141C.

Length of pedicel.—4-15 mm.

Color of pedicel.—RHS 141C.

Number of capitula per inflorescence.—5-8.

Number of disc florets per capitulum.—50-85.

Number of bracts per capitulum.—15-20.

Length of bract.—2-3 mm.

Color of bracts.—RHS 137B with red tip.

Diameter of capitulum at beginning of flowering.—4-5 mm.

Color of capitulum at beginning of flowering.—RHS N71A.

Diameter of capitulum at full flowering.—4-5 mm.

Color of capitulum at full flowering.—RHS 059A.

Length of stigma and style.—4-6 mm.

Color of stigma.—RHS 059A.

Fragrance.—No fragrance.

Lastingness of the bloom.—The capitula of one umbel open over a period of 3-4 weeks.

Seedset.—‘Agmontis’ has seedset.

Seed:

Shape.—Pentagonal.

Length.—2 mm.

Diameter.—0.8 mm.

Color.—RHS 202A.

Pappus.—Very short.

Roots:

Type of roots.—Fibrous. Roots start to grow on every part of the stem that contacts the soil, so not only at the nodes.

Physiological and ecological characteristics: Good tolerance to heat and cold, but no frost tolerance. Strong resistance to pests and diseases.

What is claimed is:

1. A new and distinct variety of Ageratum plant, substantially as illustrated and described herein.
